(19) United States
(12) Patent Application Publication
(10) Pub. No.: US 2021/0307652 A1
(43) Pub. Date: Oct. 7, 2021

(54) SYSTEMS AND DEVICES FOR MEASURING, CAPTURING, AND MODIFYING PARTIAL AND FULL BODY KINEMATICS

(71) Applicants: David Lari, San Francisco, CA (US); Luke Clauson, Redwood City, CA (US); Mike Schaller, Redwood City, CA (US)

(72) Inventors: David Lari, San Francisco, CA (US); Luke Clauson, Redwood City, CA (US); Mike Schaller, Redwood City, CA (US)

(21) Appl. No.: 14/880,165

(22) Filed: Oct. 9, 2015

Related U.S. Application Data
(60) Provisional application No. 62/061,793, filed on Oct. 9, 2014.

(51) Int. Cl. A61B 5/11; G06N 7/00; G06N 20/00; G06F 3/01

(52) U.S. Cl. CPC A61B 5/1122; G06F 3/011; G06N 20/00; G06N 7/005

(57) ABSTRACT

Devices, systems, and methods are disclosed for measuring, capturing, and modifying the motion of a body, for providing visual comparisons, metrics of comparison, and for generating motion standards. The motion standards may be generated during motion capture and may be used as references for comparing amongst other motion standards or captured motions. Comparisons may generally be used for training and improving upon motions, such as athletic related motions, rehabilitation related motions, and the like. In certain embodiments data capture is done primarily by sensor units worn on a body which communicate amongst each other and are supplemented by probabilistic relationships and models which infer or modify motion not captured by sensor units, which when paired with a secondary processing unit analyzes the data and provides a means of comparing and modifying a captured motion, and which stores or relays such data to a tertiary device, such as a remote database.

### CROSS-REFERENCES TO RELATED APPLICATIONS

This application claims priority to U.S. provisional patent application No. 62/061,793, filed on Oct. 9, 2014, the full disclosure of which is incorporated herein by reference.

### FIELD OF THE INVENTION

This disclosure relates generally to wearable sensing devices and particularly to a system and method for measuring, capturing, and modifying a motion profile using wearable devices.

### DESCRIPTION OF THE RELATED ART

Wearable technology (“wearables”) has seen dramatic growth in recent years—for instance, some estimates show wearable smart bands (products from FitBit, Jawbone, Nike, Swift) alone heading towards 350% growth in 2014, with shipment projections of 8 million units for the year. Wearables have proven their worth in providing users with a general indicator of activity, and in providing data and a means for historical comparison with some level of metric tracking. Another class of wearable sensor such as the devices from Notch, Xens, Radio6ense, Invensense, APDM, Biosens, Apple, Captapult Sports etc. track wearer's (including team members') motion and are being used for motion- and other user metric-tracking for movie making, video gaming, virtual reality, sports motion recording, team coordination, physical therapy, surgical recovery etc. These are a natural extension of the wired and more bulky wireless sensors used in university kinematic motion labs for many decades. This class of wireless wearable sensor is often relatively compact, wireless, communicates from sensor to sensor and to a host and often has real time data processing on board. All the products of the above listed companies have the ability to track and/or describe motion in some capacity. Some allow for motion data capture (APDM, etc.), though none have capability of modifying captured motion, nor do any have ability to dynamically compare a modified motion profile to a recorded motion in real-time.

None of the systems have the ability to perform motion profile modification. Thus there is a need for improvement in existing systems and products.

### SUMMARY OF THE INVENTION

Systems and devices for measuring, capturing, and modifying an individual's partial or full body kinematics, by at least one wearable sensor, are disclosed. The system comprises at least one sensor configured to transduce and provide kinematic information about a movement of the user's body. The sampling rate of a motion may be dynamic, such as to collect more data points during rapid and/or critical motions, and less during slower and/or less critical motions. The system further comprises a computer interface device with processor memory and a display configured to analyze the kinematic information about said movement. The computer interface device is configured to store a pre-determined motion standard, provide a means of modifying a captured motion, and compare the kinematic information to said motion standard, and output the results of the comparison through the display.

In embodiments of the system the sensor is a gyroscopic sensor, an accelerometer, a strain sensor, a resistive, capacitive or a pressure sensor. In one embodiment of the system the motion standard is determined by recording the user's own movement. In one embodiment of the system the motion standard is determined by manual or automatic programmatic manipulation of a recorded movement. In one embodiment of the system the motion standard comprises a database of other users' movement. In one embodiment the motion standard is peer-to-peer shared between users. In some embodiments of the system the analyzing kinematic information compensates for body differences between that of the motion standard and that of the user.

In various embodiments of the system the computer interface device comprises a mobile phone, a laptop computer, or a wearable computer. In various embodiments of the system the sensors communicate to the computer interface device via wired or wireless communication. In some embodiments the wireless communication uses a wireless communication protocol selected from one of radio frequency, Bluetooth, Zigbee, Ultra Wide Band or WiFi.

A method of measuring, capturing, and modifying a user's body kinematics is disclosed comprising receiving inputs from sensors relating to movement of a user, analyzing the sensor inputs to determine kinematic state information of the user, comparing the kinematic state information to that of a motion standard, and representing the result of the comparison to the user via an interface. In one embodiment of the method the motion standard comprises a database of the user's own movement. In one embodiment of the method the motion standard comprises a database of movement of individuals other than the user. Captured or modified motions may be used for gamification—in the form of either generating avatars that mimic the motion capabilities of a user and allowing users to compete virtually, or in the form of users competing against each other or against artificial motions by direct comparison of motion standard metrics.

In various embodiments of the system sensors worn by the user to track motion may communicate with each other to automatically scale the user. Additionally, sensors may be placed in nests intended for placement on various parts of the user's body, which allow the sensors to automatically infer their placement on the user's body.

In various embodiments of the system sensors worn by the user to track motion may be insufficient to capture the desired body kinematics—whether partial or full, in which case probabilistic/computational modeling, statistical techniques, machine learning, or the like may be utilized to create an optimized motion profile by either directly optimizing the motion profile of partial body kinematics, or by inferring the motion which was not captured by sensors to create a more descriptive motion profile, such as inferring full body kinematics from partial body sensor input.

### BRIEF DESCRIPTION OF THE DRAWINGS

The invention has other advantages and features that will be more readily apparent from the following detailed description of the invention and the appended claims, when taken in conjunction with the accompanying drawings, in which:

FIG. 1 shows one embodiment of a system for automatic detection of sensor location by means of a sensor nest.

FIG. 2 shows one embodiment of a system for scaling of a user's avatar by means of a baseline measurement.

FIG. 3 shows one embodiment of a system where various aspects of a motion are used for gamification in the form of comparing motion profiles among users or feeding user's motion profile information into a customer player in a video game.

FIG. 4 shows one embodiment of a system whereby data sampling rate is dynamic, based on optimizing sampling during times of greater motion activity.

FIG. 5 shows one embodiment of a system whereby a user may select to compare themselves to another under various conditions.

FIG. 6 shows one embodiment of a system whereby a network of sensors communicate amongst each other to automatically scale a model avatar.

FIG. 7 shows an example of a region specific subset of sensors overlaid on an outline of a human body (left), as well as a reduced region specific subset of sensors overlaid on an outline of a human body (right), whereby probabilistic modeling or the like may be used to estimate or infer the positions of the missing sensors.

### DETAILED DESCRIPTION

### Definitions

Kinematic information: Of or relating to any metrics, descriptors, or the like which describe a motion of a physical body, in part or in whole, qualitatively or quantitatively.

Partial body kinematics: Of or relating to any motion which generally describes all primary motion of a subset of a physical body, i.e. motion of a subset of a body's comprising elements, where comprising elements are generally defined as section of a body next to or between joints, linkages, or equivalent.

Full body kinematics: Of or relating to any motion which generally describes all primary motion of an entire physical body, i.e. motion of most of a body's comprising elements, where comprising elements are generally defined as section of a body next to or between joints, linkages, or equivalent.

Motion standard: A path (or collection of paths) of a point (or collection of points) through space and time, as defined by a series of space-time coordinates, or equivalent, which generally describe the partial or complete motion of a body and/or of a body's comprising elements, and which generally, but not necessarily, are linked with specific motion activities.

A motion standard may be derived from an unaltered motion of a captured motion, a completely artificial motion defined empirically/algorithmically/manually, or the result a combination of the two, such as manipulation/modification of a captured motion. Modification/manipulation of a motion may come in the form of adjusting various coordinates and/or time points in a motion standard, or a drag and drop system of a wireframe or equivalent model of a motion standard at various parts of the motion standard, or equivalent, all of which may have the ability of software to algorithmically smooth the data. A stored motion standard may be displayed alongside a video capture of a similar motion being performed, with software algorithmically determining when and where to overlay the captured data (i.e. side by side or a direct overlay). Captured data which is similar to or used for creating a motion standard may be used to feed into such a motion standard, for further dynamic modification or manipulation, artificially by the software or manually.

Motion standards which may be individually defined, or downloaded from a database are disclosed. When performing physical activities, technique plays a large role in an individual's performance and health, which can which can be adjusted properly with the right information by repetitive motion training. This repetitive motion training is often required to perfect one's athletic form. For example, the Notch device can be used to record a motion then provide haptic feedback through the sensor to the user based on programmatic decisions surrounding the motion. Their solution however, does nothing to compare a user's motion data to an external standard. No predicate system allows dynamic motion feedback against a professional athlete's motion or against a motion recorded under, and possibly modified under the eye of a trainer or coach. Systems such as the Notch could ostensibly be used as motion training tools, like many other similar devices, but no devices support, currently allow or purport to allow dynamic comparison and feedback of motion compared to an external modified motion standard. Such a baseline can either be a trained motion, or a programmed motion. Such as device may additionally monitor electro-physiological signals, as well as body heat, perspiration, etc. which could be programmatically used to further assess performance or conditions. When multiple sensors/gauges are used in concert, they may communicate with one another, additional sub-sensors such as in-shoe sole strain gages etc. or data capture devices or arrays for more systematic measuring and tracking.

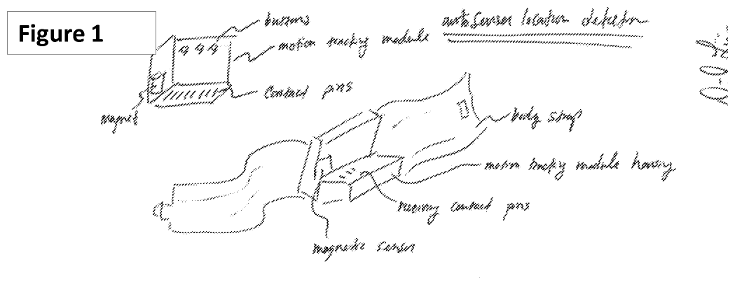

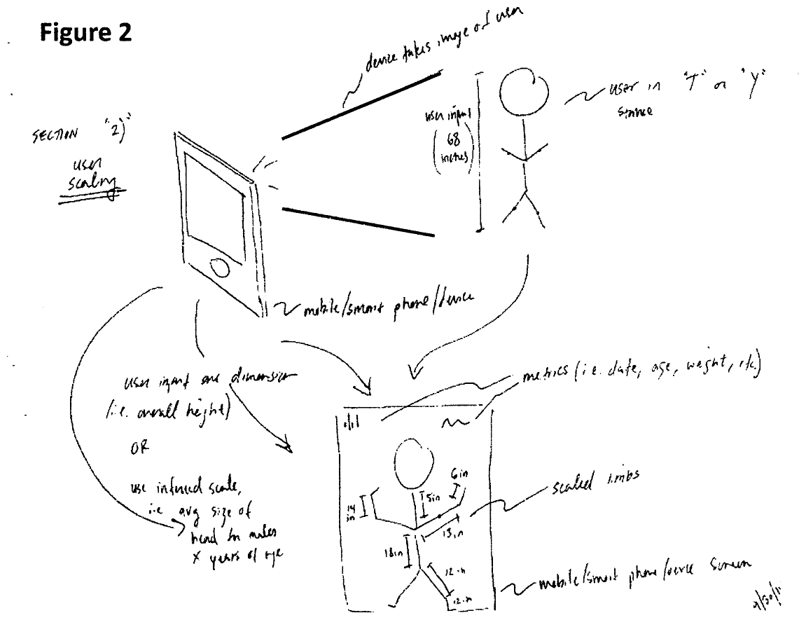

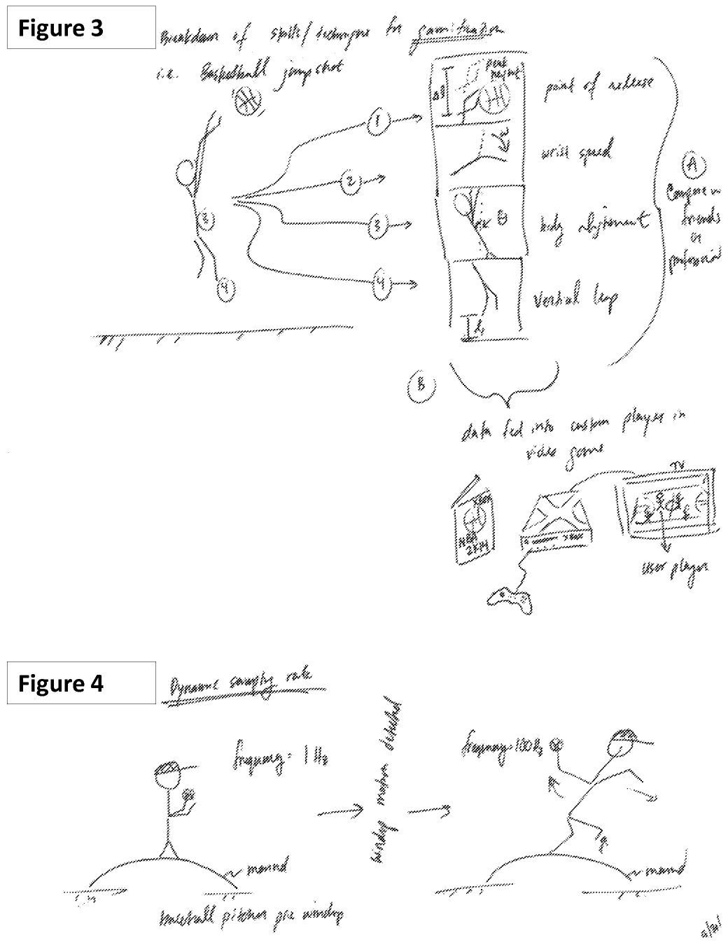

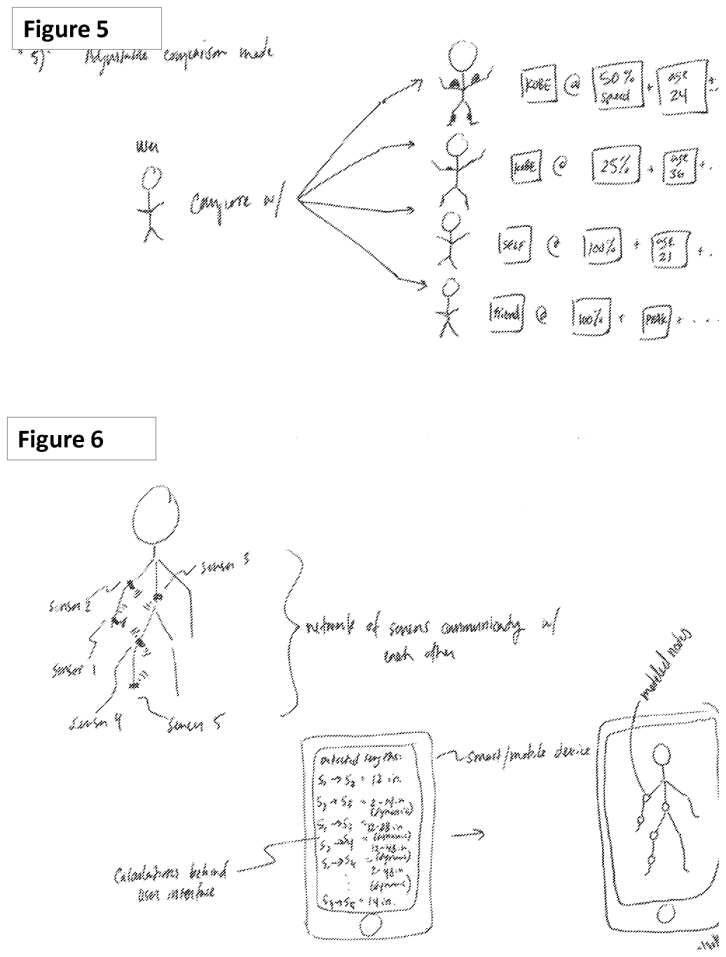

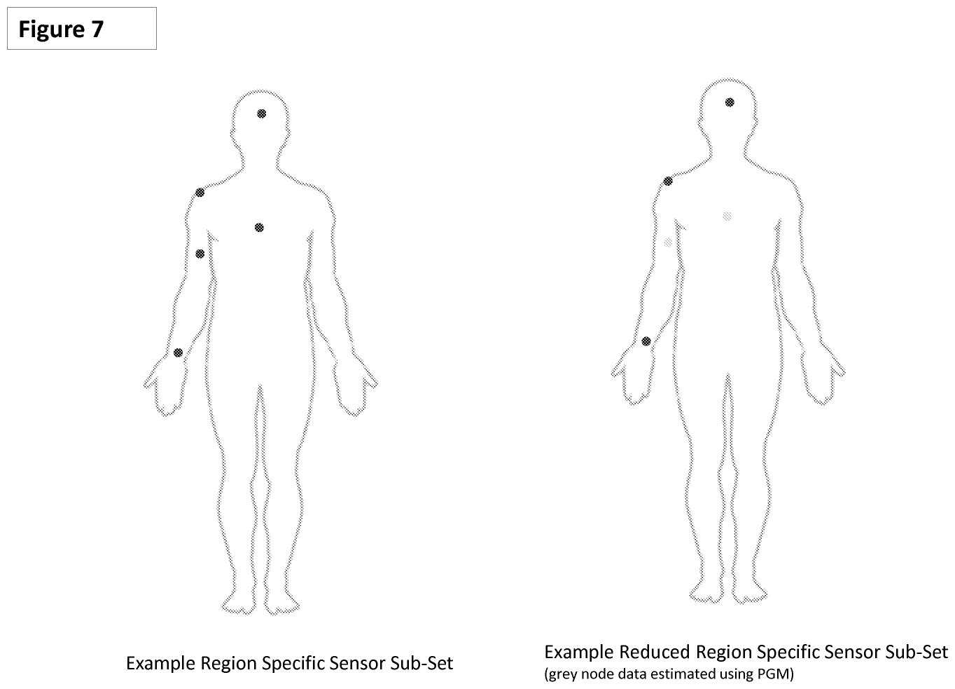

The invention discloses devices, systems and methods for measuring, capturing, and modifying partial and full body kinematics of a user according to the embodiments set forth herein. In one embodiment, a system for achieving the aforesaid objectives is disclosed in FIG. 1A. As shown in FIG. 1A, the system 100 comprises wearable sensor unit 110 worn by a user H, that communicates with a secondary computing device 130 either directly or via a primary communication device 120. Wearable sensor unit 110 is configured to transmit data to communication devices 120 and 130, either through wired or wireless means. Data from sensor unit 110 captured by computing device 130, is configured to be dynamically modified or manipulated, artificially by the software or manually by the user. Optionally, the computing device 130 may communicate with the Cloud for accessing data or to an additional computing and display device such as a laptop 140.

Method & apparatus for providing automatic recognition of placement of a sensor based on sensor receiving nests are described herein. Such a system may comprise a nest, such as a harness, having built in communication means with the sensor to determine sensor location and presence. The means are preferably contactless to avoid degradation and faulty connections. This allows the sensor to be generic and improves flexibility in the system. The same sensors would then be usable on various parts of the body. Such self-recognition additionally reduces the complexity by keeping the sensors highly modular. In the event of device failure, having generic sensor placement facilitates simple replacement. A benefit to this system is not having a calibration step in the system because the sensors will be able to identify where they are on the body by assuming the recognition of the harness and sensor, and may communicate with each other to determine distances between the sensors. Recognition can based on contact methods (buttons, or combinations of pins which are populated to close a circuit and each harness has different set of pins populated) or contactless methods (embedded magnet of different strength or position in the harness (i.e. PU molded holster) and the magnetometer on the sensor reads the magnetic field and it is pre-programmed with which magnet strengths correspond to each harness so the software knows how to identify the sensor with a body segment) RF ID, etc.

A method of automatic scaling of a 3D avatar human body model for using 3D motion capture and analysis with biometric inputs is further described herein. Method for accepting height and weight values to scale a generic human body model taken from statistical data. This allows for more accurate analysis of data and for more meaningful and accurate comparisons between data from different users. Manual entry for more specific data, such as limb lengths may supplement the captured data. In one instance of this method, a picture of a person standing in with arms extending out horizontally (i.e. body is in T-shape, or equivalent such that various key body dimensions can be captured), may be taken to scale limb lengths based on height input, such information is further useful for acquiring all limb to joint, and equivalent, lengths. Additional methods include using an image of the person for scaling the model by means of automatically detecting limb lengths and comparing to predictive values, and not requiring the user to enter any data at all. Any combination of the above two methods may be used as well.

While motion capture may be used for determining analytics critical for comparing, tracking, and predicting motion, it may also be used for gamification. Each skill set or technique associated with a captured motion or motion standard may be broken down into multiple more basic drills or skill sets that build to the final result, i.e. the perfect jump shot (basketball) or the perfect forehand (tennis). These drills or segments of the technique are arranged in a series that build on one another to increase complexity of the movement as coordination improves, similar to technique used for teaching athletics (i.e. fundamentals). The application can allow players to compare themselves with their professional idol (or friend, trainer, target hypothetical motion model etc) immediately, but there is also the option to get better by, for example, actually seeing where they are going wrong or deviated from a desired motion standard. If certain parts of the technique are very good, user could begin at a certain segment, i.e. “the jump” of a jump shot wherein that segment looks at jumping form or the like. The gamification comes in to play as each segment requires a certain level of proficiency to progress to the next stage, proficiency either in consistency or accuracy or both, and there may be rewards and prizes (i.e. in the form of virtual score, coupons, encouragement messages, access to digital content, etc.) at the end of each stage or when something good is noticed (or even when the user has used the service for a predetermined amount of time or logins). Prizes or rewards for proficiency or segment completion could additionally be secret tips from that pro on that technique or something about that technique that would not be easy to notice (such prizes may be available for a limited time, such as a one-time viewing of a message from a professional athlete on technique, to make content more valuable/rare and prevent users from sharing such prizes). Auxiliary drills to practice awarded techniques, or the like, may be made available to the user as well (for example, if a certain technique revolves around a head-fake, then there could be a bonus drill that attaches a sensor to the head to see how your head-fake compares with an athlete of choice (i.e. Kobe Bryant, or the like)). Other rewards (may be in the form of a surprise) could be a prerecorded (or real) call from the star who they are emulating giving them some encouragement or advice and shows up as the athlete (i.e. Kobe Bryant, or the like) calling and has pre-recorded voice hints (if call is prerecorded). Another bonus may be activating a mode such as a “pressure player” mode whereby instead of waiting until the end of the session to give feedback or interact with the user, the system is actively predicting or introducing distractions into certain and possibly random times in the movement to disrupt the player and make the drill more challenging. “Pressure player” mode can be activated for a limited period of time by doing something special or just using the system enough, and would go away and need to be earned again. This mode could also have a user-base wide challenge for that technique that lasted a limited time. Another method of gamification could be having the system link up with a video game console game (i.e. Xbox, Playstation, PC, etc.) where the power/strength/skill of the player in that game is determined by how proficient the player is in real life at their relevant skills. Real life data therefore, would translate to the virtual world. For required data not available from the sensor, either default or user entered metrics could be used. Players may form teams (individual or group) of avatars of themselves and/or friends and challenge other users or see how they stack up against a professional team, or the like. A related embodiment and application of this would be for recruiting purposes. A coach, recruiter, or the like, may integrate potential recruits into a virtual version of the current team (also could be used for past or future teams) they are coaching, and analyze how that user would integrate with the rest of the team. Such an analysis may be done by comparing stats, running simulations, or the like.

In some instances, a motion capture sensor may detect the activity or motion being performed, and based on the speed, acceleration, etc., the sensor may monitor data at an optimal rate (i.e. more frequent during fast, rapid motions, less frequent for slow motions). For example, in golf the back swing is generally slower than the downswing so sampling frequency can be reduced during this phase, but the downswing and motion through the ball will be fast so sampling frequency can be increased during this phase. Such a feature may help optimize battery power and transmission speed. During periods of no movement, the sensor may be set to not sample at all.

Various modes of motion analysis may be accessible to the user to optimize a desired change in motion. For instance, in a mimicking type mode, a user's form may be directly compared to an athlete, friend, trainer, etc. In such a mode one could adjust the speed of the person being compared with by a certain percentage so the user could set a goal and compare themselves to a professional performing at a certain percentage of their peak or normal performance. This performance offset could be adjusted manually, or algorithmically, to allow the user to gradually build up to comparing with a professional athlete at 100%, or any level they choose. If a user's full body kinematics is captured, complete direct comparison to a master motion standard is easily applicable. In the case that a user is under-sensored partial comparison is possible. For example, a user's actual hand motion could be married with the forearm and upper arm motion from the master model to create a more realistic visual motion representation, one that includes their part and the master motion's part. These parts may be used individually or in a combined manner to reduce the number of sensors required. Learning models may include machine learning techniques such as probabilistic graphical models, neural networks, support vector machines and nonlinear regression algorithms. One or more of these learning models can be used to determine the mathematic relationship between the motions and positions of all of the nodes in the network. One skilled in the art can recognize that any number of other mathematical, computational, and probabilistic systems can be used. This can be done on both master motion standards as well as dynamically provided data. The system can create a model of the output from each sensor based on the output of all of the other sensors in the network. If one or more sensors are removed from the network, the system can use the learned network relationships to estimate the state of the missing sensors. The removal of the sensor may also be unintentional due to sensor failure, communications dropout or the like. In this case, the network sensor state estimation also provides fault tolerance.

Further, the “gamification” of this type of usage could be accomplished by breaking down a user's motions as compared to the part of the master motion to which they are being compared. Specifically, a master motion may comprise information for full body kinematics, but the user may have a subset of sensors which only cover partial body kinematics. In this case, they may compare various partial body kinematics to a master, such as head motions, forearm motions, etc., up to and including full body kinematic motion.

In the event multiple sensors are worn by the user, a length sensing mechanism could detect the distance between sensors for more accurate modeling of the body automatically, such as embedded variable resistor or piezoelectric sensor in a garment, mount, holder, etc. that senses the distance between two or more sensors. Such a system could use a piezoelectric material or pressure gauge to sense changes in force which would translate to a change in length or distance.

Additionally, what may be included in an ideal embodiment of the system:

Motion database of standardized drills, motions, forms, etc. for identification of talent early. Users would likely be agents, recruiters, scouts, colleges, sponsors, etc. In one embodiment, the customer may search for specific criteria of specific motions (i.e. fastest acceleration off the blocks for runners, or ambidextrous pitchers (pitchers that throw with either arm) that can throw with a certain velocity from either arm).

Features for integration with broadcasting. Motion capture data/graphics on screen drawing tool for looking at player positions during commercials or half time, or between events to make viewing more interesting.

Features directed at sports medicine, such as monitoring joint angle(s) dynamically, in home exercise performance with motions sent to trainers to look so they don't have to always be with the player, etc.

Integrated with the baseline comparison tool may be a tool for adjusting an acceptable error band by the user or optimized algorithmically. For instance, a larger band may be used when a user wants to detect improvement early on, and then tightened as the skill, motion, etc. is improved upon. An adjustable error band (alternately called a “likeness rating”) allows feedback to be relevant at all skill levels and adaptable to the specific skill level. The error band could be automatically adjustable so that if after 10 repetitions, 9 were good, the error band will narrow further to make the challenge more difficult. This adjustment could be set in software by an algorithm or selectable by the user. Error gaps can be set to specific segments of a movement or technique. For example a basketball shot has an initial body position, a pre-jumping stance, peak jump stance (shooting position), a release stance, and a follow through stance. Each one of these segments of the movement can have its own error gap associated with it and the movement can be broken up into these different discrete sections.

In addition to error band adjustability, the movements, motions, etc. may also be broken down into discrete sections (i.e. in many cases a complete motion of interest may comprise many sub-motions or stages of motion). This allows for each stage to be analyzed separately, providing targeted and specific feedback. Previous technologies (i.e. Notch and Vibrado) that only buzz or vibrate if something went wrong or right do not give any useful information about what part of the motion was good or bad. In most cases it is one or two aspects of a technique or motion which are causing the problem and need the work, but analyzing an entire motion and giving a single point of feedback on the motion as a whole may not tell the user how to correct an incorrect or undesired motion. On a screen this could be signified by an image staying green as long as the motion is within the error gap, going yellow to red at the moments when the motion is poor and outside the error gap, the tone being related to how far off the motion was. In the case of noise, a sound signifying good could stay on (or off) as long as the motion was good and change in tone as the motion degrades with the tone deepening or increasing the farther off the motion is from the target. Alternately, rather than a changing pitch tone a metronome style beat may be sounded to help with timing cadence. The beat may occur at specific points in the motion standard, corresponding with visual markers on the user interface screen. Further, voice commands may be given from a wireless speaker such as in or on an earbud, necklace, etc.

Additionally, relevant feedback may come in the form of actual instructions as to what may be done to optimize or correct a motion, in addition to a visual or auditory or equivalent indication that a motion was incorrect or non-optimal. Current technologies which vibrate or beep are not detailed in how to improve a motion. Therefore, software analysis of the motion may provide the user with a voice command, or visual, or equivalent, telling them specifically what they did wrong and how to improve it. In the case of elbow drop of a quarterback in football, if the software analyzed that the elbow drop was too long by a certain distance then it could simply inform the user to move their elbow up by the determined distance (or the software may determine the root cause to be associated with a different, related or associated motion, and recommend a change that that related or associated motion, which may affect the first incorrect motion. Users that do an activity or a motion may require instant feedback during every repetition to avoid repeating and creating bad habits but will not have the time to stop performing their activity as this slows down their progress and reduces the effectiveness of the muscle memory they are developing by repeating a motion or technique over and over. For instance, if a marathon runner started running with bad form at mile 6, it would be very damaging to run the remainder of the marathon with bad form—rather, with dynamic feedback, the runner could adjust their form actively, as they receive feedback from the device.

What is claimed is:

1. A system of measuring, tracking, and capturing a user's body kinematics comprising:
receiving measured inputs from sensors relating to movement of a user;
analyzing said inputs' data to create a partial or full body kinematic model;
parametrizing said measured kinematics model by means of a learning model;
presenting the output of the parametrization to the user via an interface.

2. The system of claim 1, wherein the learning model is used to algorithmically infer any motion of the user not directly captured via sensors.

3. The system of claim 1, wherein the location of a sensor on the user may be automatically detected by means of the nest to which a sensor is paired.

4. The system of claim 3, wherein the method of detection is near field communication.

5. The system of claim 1, wherein the captured motion is used to create a virtual avatar which depicts such captured motion.

6. The system the claim 1, wherein a Bayesian network is used to capture the learning model.

7. A system of measuring, tracking, and capturing a user's body kinematics comprising:
receiving measured inputs from sensors relating to movement of a user;
analyzing said inputs' data to create a partial or full body kinematic model;
parametrizing said measured kinematics model by means of a learning model;
using information from the parametrized model to augment the measured inputs to create a combined motion standard;
presenting the output of the combined motion standard to the user via an interface.
